(19) United States
(12) Patent Application Publication
(10) Pub. No.: US 2023/0000986 A1
(43) Pub. Date: Jan. 5, 2023

(54) MNK INHIBITORS AND EIF4E PHOSPHORYLATION MODULATION TO TREAT INFLAMMATORY PAIN IN THE AGED

(71) Applicant: Board of Regents, The University of Texas System, Austin, TX (US)

(72) Inventor: Michael D. BURTON, Richardson, TX (US)

(73) Assignee: Board of Regents, The University of Texas System, Austin, TX (US)

(21) Appl. No.: 17/810,404

(22) Filed: Jul. 1, 2022

Related U.S. Application Data
(60) Provisional application No. 63/217,713, filed on Jul. 1, 2021.

(51) Int. Cl. A61K 45/06; A61P 29/00

(52) U.S. Cl. CPC A61K 45/06; A61P 29/00; A61K 2121/00

(57) ABSTRACT

Disclosed is an important therapeutic strategy of targeting MNK to block eIF4E phosphorylation for the treatment of persistent inflammatory pain in later life. In one embodiment, a therapeutic drug that targets MNK and inhibits MNK is selected in order to block eIF4E phosphorylation and the therapeutic drug is administered to the subject with sufficient dosage to block eIF4E phosphorylation. The treatment is suitable for aged subjects experiencing inflammatory pain such as age-associated inflammation, age-associated low-grade inflammation, CFA-induced acute inflammation, spontaneous pain, mechanical hypersensitivity or thermal hypersensitivity.

### PRIORITY CLAIM

This application claims benefit of priority to U.S. Provisional Application Ser. No. 63/217,713, filed Jul. 1, 2021, the entire contents of which are hereby incorporated by reference.

### STATEMENT OF GOVERNMENT SUPPORT

This invention was made with government support under grant no. K22 NS096030 awarded by the National Institutes of Health. The government has certain rights in the invention.

### FIELD OF THE INVENTION

The present invention relates to eIF4E phosphorylation and corresponding modulation of pain and neuroinflammation in the aged.

### BACKGROUND

Aged individuals represent more than 20% of the US population and their numbers are predicted to increase by 150% by 2050 (Roberts et al., 2018). There is an association of neuropathological states such as inflammation, chronic pain, depression, and anxiety with normal aging (Rea et al., 2018). The molecular processes within cells deteriorate as age advances, leading to changes in overall health and behavior (Kubben and Misteli, 2017). There are higher incidences of acute pain transitioning to chronic in elderly individuals (Dahlhamer et al., 2018). The persistence of pain in the context of age-associated sustained low-grade inflammation needs to be studied to identify targets for preventives and therapy. An important molecular biological process that is involved in the manifestation of neuropathological states is translation regulation (Gonskikh and Polacek, 2017). This highly regulated process enables translation of particular mRNA subsets to mediate changes in the immune and nervous system. The inventor hypothesized that this pathway plays a critical role in acute pain and the transition to chronic pain in aging.

Protein translation is heavily regulated to limit the availability of intracellular and secreted proteins for cell-to-cell communication, extracellular process formation, and general use to the cell, based on exogenous stimuli or responses to injury. The mammalian target of rapamycin (mTOR) pathway is particularly responsive to inflammatory modulators, with downstream effects on cap-dependent translation (Johnson et al., 2013; Thoreen et al., 2012). Signaling through mTOR and the mitogen-associated protein kinases (MAPK) pathway are two parallel pathways that converge to control cap-dependent translation. mTOR can phosphorylate eIF4E binding proteins (4E-BPs) to release eIF4E for initiating translation (Joshi and Platanias, 2014). The binding of eukaryotic initiation factor 4F (eIF4F) complex to the 5′7-methylguanosine (m⁷G) cap and poly-A binding protein to the 3′ end of mRNAs marks them for translation once the 43S pre-initiation complex has been formed (Merrick and Pavitt 2018). This complex contains a scaffolding protein eIF4G, a 5′ cap-binding protein eIF4E, and a RNA helicase eIF4A for unwinding secondary structures near the 5′ cap. Binding of eIF4E to the 5′ m⁷G mRNA cap is the rate-limiting step for translation initiation (Merrick and Pavitt, 2018). MAPK interacting kinases 1 and 2 (MNK1/2) can also phosphorylate eIF4E directly, which can affect its binding to the mRNA 5′ cap (Buxade et al., 2008; Scheper et al., 2002). After exposure to an inflammatory stimulus or injury, MNK1/2 are activated in response to various pro-inflammatory cytokines and mediate translation of selective mRNA subsets that are involved in modulating pain and inflammation (Khoutorsky and Price, 2018; Megat et al., 2019). An upregulation of algogens such as bradykinin, tumor necrosis factor alpha (TNFa), interleukin 1 beta (IL-1β), and interleukin 6 (IL-6) has been shown to mediate pain states and circulating levels are reported to be higher in aged individuals (Ellis and Bennett 2013; Cruz-Almeida et al., 2015).

Inflammatory mediators have been shown to sensitize peripheral nociceptors, which in turn engender changes in the cells of the central nervous system (Pinho-Ribeiro et al., 2017). With age, the inflammatory response is enhanced and a higher level of cytokines, chemokines, and other signaling molecules are produced from already primed and activated glial cells (Ye and Johnson, 1999; Burton and Johnson, 2012; Dilger and Johnson, 2008; Frank et al., 2010; Norden and Godbout, 2013). Persistent inflammation within the peripheral nervous system leads to pain sensitization which can be driven by plasticity in peripheral nociceptors and through central sensitization (Rea et al., 2018; Cruz-Almeida et al., 2015; Dilger and Johnson, 2008; Frank et al., 2010; Chung et al., 2019; Gubbels Bupp et al., 2018; Price and Gold, 2018). There is evidence that aged microglia in the brain also secrete higher levels of pro-inflammatory cytokines, which in turn mediate changes in pain states and behavioral plasticity (Ye and Johnson, 1999; Norden and Godbout 2013; Garner et al., 2018; Mangold et al., 2017; Perkins et al., 2018).

Studies using MNK1/2 knock-out mice have elucidated the differential contribution of each kinase to phosphorylation of eIF4E and suggest that eIF4E regulation by MNK phosphorylation does not influence cell survival and growth (Ueda et al., 2004). The eIF4E^{s209A} mouse model has an alanine substituted for serine at position 209 of eIF4E, which renders it resistant to phosphorylation by the MNKs (Furic et al., 2010). This animal model has been used to demonstrate the role of eIF4E phosphorylation-mediated translation in nociception and inflammation in adult mice (Megat et al., 2019; Amorim et al., 2018a; Herdy et al., 2012; Moy et al., 2017; Moy et al., 2018a; Shiers et al., 2019). The progression of pain and the role of the immune response in age-associated inflammation is yet to be investigated. While the inventor originally hypothesized that modulation of eIF4E over the lifespan would have anti-inflammatory effects, recent data has revealed that the young adult eIF4E^{S209A} model shows an upregulation in a subset of pro-inflammatory mediators (Amorim et al., 2018b; Aguilar-Valles et al., 2018). Upregulated levels of pro-inflammatory cytokines interleukin 2 (IL-2), interferon gamma (IFNγ), and tumor necrosis factor alpha (TNFα) have been reported in the naïve brain and plasma of eIF4E^{S209A} mice and these levels were shown to be exaggerated following peripheral lipopolysaccharide treatment compared with wild-type animals (Amorim et al., 2018b). Conversely, silencing of MNKs in the presence of lipopolysaccharide treatment has been shown to reduce expression of TNFα in mouse macro-phage cultures (Rowlett et al., 2008).

The descriptions of the various embodiments of the present invention have been presented for purposes of illustration, but are not intended to be exhaustive or limited to the embodiments disclosed or claimed. Many modifications and variations will be apparent to those of ordinary skill in the art without departing from the scope and spirit of the described embodiment.

The terminology used herein was chosen to best explain the principles of the embodiment, the practical application or technical improvement over technologies found in the marketplace, or to enable others of ordinary skill in the art to understand the embodiments disclosed here.

### BRIEF SUMMARY

The aged population has a higher probability of developing chronic pain from acute insults because of age-associated low-grade inflammation. Several emerging studies have shown a crucial role of cap-dependent translation in the development of chronic pain in young adult animals; however, its role in the aged has never been reported. Acute and chronic inflammatory responses, including pain, are altered over age, and understanding how cap-dependent translation can represent an important and druggable pathway is imperative for understanding its therapeutic potential. Here, the inventor tested how an inflammatory stimulus, complete Freund's adjuvant (CFA), affects spontaneous and evoked pain, as well as inflammation in young versus aged mice that lack functional cap-dependent translation machinery (eukaryotic translation initiation factor 4E (eIF4E)) compared with age-matched wild-type (WT) mice. Interestingly, the inventor found that CFA-induced acute pain and inflammation are modulated by eIF4E phosphorylation in aged but not young animals. Aged transgenic animals showed attenuated paw temperature and inflammation, as well as a mitigation in the onset and quicker resolution in mechanical and thermal hypersensitivity. The inventor found that levels of interleukin (IL)-1β and tumor necrosis factor (TNF)-α are elevated in dorsal root ganglia in aged WT and eIF4E transgenic groups, despite faster resolution of acute inflammation and pain in the aged eIF4E transgenic animals. The inventor proposes that these cytokines are important in mediating the observed behavioral responses in the young and represent an alternate pathway in the development of age-associated inflammation and behavioral consequences. These findings demonstrate that eIF4E phosphorylation can be a key target for treating inflammatory pain in the aged.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS AND TABLES

The novel features believed characteristic of the illustrative embodiments are set forth in the appended claims. The illustrative embodiments, however, as well as a preferred mode of use, further objectives and features thereof, will best be understood by reference to the following detailed description of illustrative embodiments of the present disclosure when read in conjunction with the accompanying drawings and brief descriptions thereof.

FIG. 1 is a flow chart for the treatment method in accordance with the present invention.

FIGS. 2a-2d—Timeline of the behavioral tests performed and validation of mutant mice. FIG. 2a—All cohorts of mice were acclimated to the behavior testing apparatus for 2 days, 4 h each, before starting experiments. The baseline readings for grimacing behavior, paw withdrawal in response to von Frey filaments, paw withdrawal in response to heat stimuli, paw temperature, and paw edema were recorded before injection of CFA into the left hind paw, which was designated as the ipsilateral paw. Experimental readings for the affective behaviors were recorded at 4 h, day 1, 2, 3, 5, and 7 after the injection. CFA, complete Freund's adjuvant. FIG. 2b—Representative agarose gel showing genotypes of WT, eIF4ES209A−/−, and eIF4ES209A+/− mice. FIG. 2c—eIF4E protein is not phosphorylated in the eIF4ES209A cohorts. FIG. 2d—Total eIF4E protein levels in tested tissues from all cohorts show no differences across age and genotypes.

FIGS. 3a-3f—Inflammation at the injection site is resolved faster over 7 days in aged eIF4ES209A mice compared with WT. Graphs depicting the paw temperatures recorded by FLIR at indicated times post CFA injection in young (FIG. 3a) and aged (FIG. 3b) mice. Data plotted as means with standard error (young WT, n=7; young eIF4ES209A, n=10; aged WT, n=21; aged eIF4ES209A, n=8). The dashed line indicates the mean temperature of the contralateral paws from all experimental animals on that graph. Two-way repeated measures ANOVA with Sidak's post hoc was performed. ns, not significant, ****p<0.0001. FIG. 3c -Effect size for all four cohorts. Two-way ANOVA with Tukey' s post hoc test was performed for multiple comparisons between genotypes for young and aged animals. **p 0.0043, ***p 0.0005. Graphs depicting the volume of water displaced from the plethysmometer due to paw edema at indicated times post CFA injection in young (FIG. 3d) and aged (FIG. 3e) mice with effect size for all 4 cohorts (Figure 3f). The dashed line indicates the mean volume of water displaced by the contralateral paw. Data are plotted and analyzed as stated for parts FIGS. 3a, 3b, and 3c. For FIGS. 3d and 3e, ns, not significant, ***p 0.0009, ****p<0.0001. For FIG. 3f, **p 0.0087. BL, baseline; 4 h, four hours; D1, day 1; D2, day 2; D3, day 3; D5, day 5; D7, day 7; CFA, complete Freund's adjuvant; FLIR, forward-looking infrared.

FIGS. 4a-4c—Aged eIF4ES209A demonstrate reduced spontaneous pain. Graphs depicting grimace at indicated times post CFA injection in young (FIG. 4a) and aged (FIG. 4b) mice. Data are plotted as means with standard error (young WT, n=7; young eIF4ES209A, n=10; aged WT, n=21; aged eIF4ES209A, n=8). For FIG. 4a and FIG. 4b, two-way repeated measures ANOVA with Sidak's post hoc test was performed. ns, not significant, **p=0.0066. FIG. 4c Effect size for all four cohorts compared with baseline. Two-way ANOVA was performed with Tukey' s post hoc. *p 0.0191, ****p<0.0001. BL, baseline; 4 h, four hours; D1, day 1; D2, day 2; D3, day 3; D5, day 5; D7, day 7; CFA, complete Freund's adjuvant.

FIGS. 5a-5f—Aged and young eIF4ES209A mice show faster resolution of evoked pain. Graphs depicting ipsilateral paw withdrawal in response to Von Frey filaments at indicated times post CFA injection in young (FIG. 5a) and aged (FIG. 5b) mice. Data are plotted as means with standard error (young WT, n=7; young eIF4ES209A, n=10; aged WT, n=21; aged eIF4ES209A, n=8). The dashed line represents the mean contralateral paw withdrawal for filament applied for all experimental animals. Two-way repeated measures ANOVA with Sidak's post hoc test was performed for multiple comparisons between genotypes for young and aged animals. ns, not significant, ***p=0.0003, ****p<0.0001. FIG. 5c—Effect size for all four cohorts. Two-way ANOVA was performed with Tukey's post hoc for multiple comparisons between age and genotypes. **p 0.0046, ****p<0.0001. Graphs depicting paw withdrawal latencies in response to heat applied at ipsilateral paw at indicated times post CFA injection in young (FIG. 5d) and aged (FIG. 5e) mice. Data plotted and analyzed as stated for FIGS. 5a and 5b. ns, not significant, *p 0.0177 for young mice, *p 0.0153 for aged mice. The dashed line represents the mean contralateral paw withdrawal latency in response to heat applied for all experimental animals. FIG. 5f—Effect size for all four cohorts compared with baseline. Data plotted and analyzed as for FIG. 5c. *p 0.0417 for eIF4ES209A mice and **p 0.0376 for aged mice. BL, baseline; 4 h, four hours; D1, day 1; D2, day 2; D3, day 3; D5, day 5; D7, day 7; CFA, complete Freund's adjuvant.

FIGS. 6a-6b—Levels of IL-1β in indicated tissues. FIG. 6a—Representative western blot for IL-1β in ipsilateral DRGs and its densitometric analysis. Data are represented as mean and standard error of the mean (young WT and eIF4ES209A, n=5; aged WT and eIF4ES209A, n=7). A two-way ANOVA was performed followed by Tukey's post hoc for multiple comparisons. *p=0.0264. FIG. 6b—Representative western blot for IL-1β in the spinal cord and its densitometric analysis. Data plotted and analyzed as for FIG. 6a (young and aged WT, n=7; young and aged eIF4ES209A, n=6). **p=0.0064.

FIGS. 7a-7b—Levels of TNFα in indicated tissues. a Representative western blot for TNFα in ipsilateral DRGs and its densitometric analysis. Data are represented as mean and standard error of the mean (young WT and eIF4ES209A, and aged eIF4ES209A n=6; aged WT n=7). A two-way ANOVA was performed followed by Tukey's post hoc for multiple comparisons. *p=0.0216. b Representative western blot for TNFα in the spinal cord and its densitometric analysis. Data plotted and analyzed as for FIG. 7a (youngWT, n=6; young eIF4ES209A, n=5; agedWT, n=7; aged eIF4ES209A, n=6). **p=0.0041.

FIGS. 8a-8b—Levels of TRPA1 and SK2 in ipsilateral DRG. FIG. 8a—Representative western blot for TRPA1 in ipsilateral DRGs and its densitometric analysis. Data are represented as mean and standard error of the mean (young WT, young eIF4ES209A, and aged eIF4ES209A n=6, aged WT n=7). A two-way ANOVA was performed followed by Tukey's post hoc for multiple comparisons. *p=0.0321 for age effect. FIG. 8b—Representative western blot for SK2 in ipsilateral DRGs and its densitometric analysis. Data plotted and analyzed as for a. No significant differences were found.

### DETAILED DESCRIPTION

The subject matter, which is regarded as the invention, is particularly pointed out and distinctly claimed at the conclusion of the specification. The foregoing and other objects, features, and advantages of the invention are apparent from the following detailed description.

To date, no one has taken advantage of the connection between eIF4E phosphorylation, aging, pain, and age-associated inflammation. Presented herein is an important therapeutic strategy of targeting MNK to block eIF4E phosphorylation for the treatment of persistent inflammatory pain in later life.

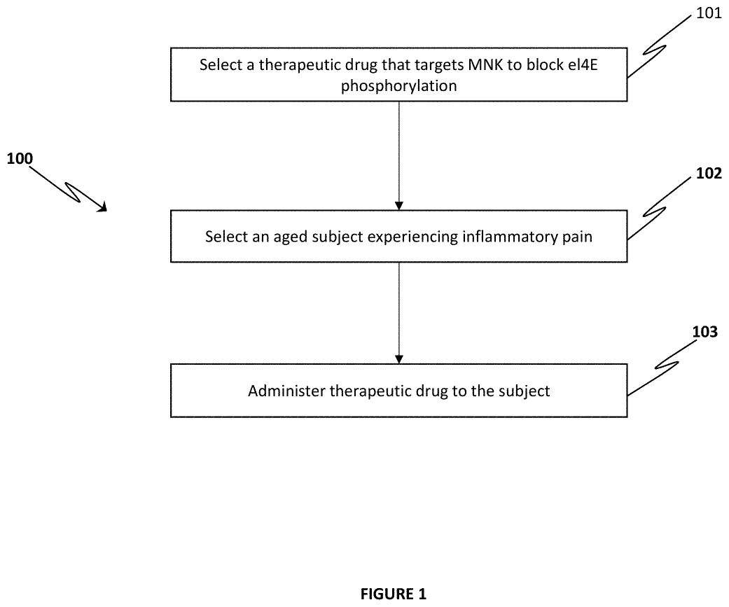

According to FIG. 1, a method of treatment for inflammatory pain in aged subjects. In step 101, eIF4E phosphorylation is blocked. In one embodiment, a therapeutic drug that targets MNK and inhibits MNK is selected in order to block eIF4E phosphorylation. Step 101 is not limited to selecting drugs that inhibit MNK, but allows for the use of other drugs that are known to block eIF4E phosphorylation. An example of a drug that inhibits MNK, suitable for the present invention, is eFT508 (Tomivosertib). In step 102, the treatment is targeted to a selected aged subject who is experiencing inflammatory pain. As used herein, “aged” means about 60% of average lifespan expectancy for the subject Inflammatory pain includes age-associated inflammation, age-associated low-grade inflammation, CFA-induced acute inflammation, spontaneous pain, mechanical hypersensitivity, thermal hypersensitivity, or a combination thereof. In step 103, the therapeutic drug is administered to the subject with dosage sufficient to block eIF4E phosphorylation and relieve inflammatory pain. In some embodiments, it is sufficient that the dosage be provided that inhibits eIF4E phosphorylation by at least 50% percentage over normal eIF4E phosphorylation in the subject, such as at least 60%, at least 70%, at least 80%, or at least 90%.

### Materials and Methods

The experimental studies in support of the present disclosure used young and aged wild-type and eIF4E^{S209A} mice, to examine eIF4E phosphorylation affected CFA-induced pain behaviors and levels of pro-inflammatory cytokines in the dorsal root ganglia (DRG) and spinal cord.

Animals. Young (6-9 months old) and aged (>22 months old) female and male eIF4E^{S209A} phospho-mutants on a C57BL/6 background were generated in the Sonenberg laboratory as previously described (Furic et al., 2010) and gifted to us. These were further bred at the University of Texas at Dallas to generate experimental animals Wild-type (WT) C57BL/6 mice were used as controls; breeders were purchased from Jackson Laboratory and bred in-house as WT counterparts for all experiments. In-house bred eIF4E^{S209A} mutant and WT mice were weaned between 3 and 4 weeks of age and tail-clipped to verify genotypes. All young mice weighed between 25 and 30 g at the time of experimental use, whereas all aged mice weighed between 30 and 40 g. Animals were group-housed in polypropylene cages maintained at 21° C. under a 12-h light/dark cycle with ad libitum access to water and rodent chow. The animal housing room lights were turned on at 6 AM and off at 6 PM daily. At the end of the study, mice were examined post mortem for gross signs of disease and any mouse found unhealthy (e.g., tumors or splenomegaly) was excluded from the dataset. Ethics approval: All procedures were in accordance with the National Institutes of Health Guidelines for the Care and Use of Laboratory Animals and were approved by the University of Texas at Dallas Institutional Animal Care and Use Committee protocols 15-15 and 16-07.

Inflammatory Insult. Complete Freund's adjuvant (CFA) was purchased from Sigma Aldrich (St. Louis, Mo.) at a 1 mg/ml concentration. Equal parts of sterile phosphate-buffered saline (PBS) and CFA were thoroughly mixed via vortexing to make an emulsion. The emulsion was also actively vortexed prior to each intraplantar injection (20 μl) to ensure equal volume injections for all animals. The working solution administered contained 10 μg of CFA. The injection was done on the left hind paw, which was designated as ipsilateral and the right hind paw was considered as a control and designated as contralateral. After injection, the following time points were measured for each animal: 4 h and days 1, 2, 3, 5, and 7.

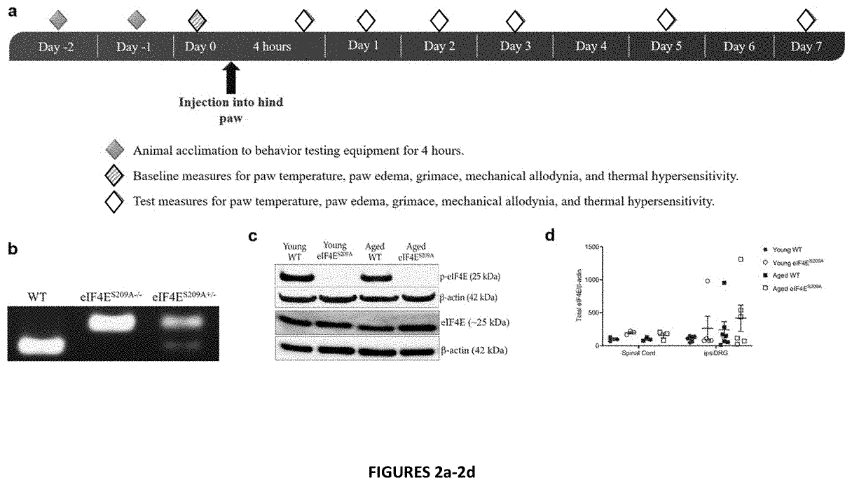

Behavioral tests. All behavioral tasks were done between 10 AM and 2 PM. Mice were acclimated to the experimental room and acrylic behavior racks for 4 h for a minimum of 2 days before any data acquisition. Spontaneous pain (Grimace Scale), paw temperature (FLIR), and mechanical allodynia (Von Frey) assessments were all conducted in the behavior racks that were 11 cm long by 10 cm wide and 4.5 cm in height. A total of 16 units were available at any given time for 16 mice to be housed individually and tested. The racks were cleaned with 70% ethanol to eliminate odor cues between each reading, baseline or experimental. Two consecutive baseline measures a day apart were taken in the behavior racks. One baseline day was taken for thermal hypersensitivity (Hargreaves) and paw edema (Plethysmometer) on the corresponding apparatus after animals baselined on the other three. Following completed baseline measures for all tests, animals were injected with emulsified complete Freund's adjuvant (CFA) at a dose of 10 μg into the left hind paw. After injection, the animals were tested at 4 h, and on days 1, 2, 3, 5, and 7 for the pain and inflammation measures listed below (FIGS. 2a-d). In order to diminish behavioral interference, tasks were done in the following order for the entirety of the experiment: spontaneous pain (Grimace), paw temperature (FLIR), mechanical allodynia (Von Frey), thermal hyperalgesia (Har-greaves), and paw edema (Plethysmometer). For mechanical allodynia, thermal hypersensitivity, paw temperature, and paw edema, readings were noted for stimuli applied to the contralateral paw and were averaged across all animals within a group. The mean value was used to provide a basis of comparison, indicated by a dashed line on the graphs and respective values. Experimenters were blind to the genotype of the animals during testing; individuals blind to the treatment and genotype analyzed data.

Assessment of spontaneous pain by grimace scale. Grimacing was assessed while the animals were in the acrylic racks before Von Frey testing. Assessment began approximately 1 h after being in the rack to ensure that the mice were calm, but not sleeping. Each mouse grimace score was measured as an average of the five components of rodent grimacing, i.e., orbital tightening or closing, ear position, whisker position, nose bulging, and cheek bulging. All five parameters measure the severity of perceived spontaneous pain. Each parameter was scored on a scale from 0 to 2, with 0 representing a normal non-painful state, 1 indicating moderate pain perceived, and 2 being representative of severe pain perceived (Langford et al., 2010).

Mechanical allodynia by Von Frey filament test. Mice were habituated to acrylic racks for approximately 1 h before testing at each experimental timepoint. Hind paw mechanical allodynia was measured by the up-down method using calibrated Von Frey filaments (Stoelting Co., Wood Dale, Ill.) as described by (Chaplan et al., 1994). Withdrawals in response to filaments up to 0.8 g were considered baseline.

Thermal hyperalgesia by Hargreaves test. Thermal hypersensitivity was measured using the Har-greaves heat plantar device (IITC Life Science Inc., Calif.) (Hargreaves et al., 1988). Mice were placed inside an acrylic enclosure on a glass surface heated evenly to 29° C., immediately after von Frey. Withdrawal latencies were measured after animals received a laser beam directed at the ipsilateral and contralateral hind paw. In order to obtain baseline withdrawal measures around 10-12 s, the inventor used the laser at an active intensity of 30%. The laser was preset to cutoff automatically at 25 s to avoid possible injury to the hind paw. A minimum of three trials of withdrawal latencies were recorded for each time point. The readings for the contralateral hind paws were averaged across all groups.

Inflammatory response tests. Paw edema measured by a plethysmometer: Paw edema was measured using the Plethysmometer apparatus (IITC Life Science Inc., Woodland Hills, Calif.). Both ipsilateral and contralateral hind paws were submerged in the water contained in the cell consistently, up to the heel of the animal throughout the experiment. The change in volume of the water level was calibrated to output a reading in milliliters (ml) shown on the electronic monitor and volume displacement was recorded.

Paw temperature recorded by forward-looking infrared camera: A forward-looking infrared (FLIR) camera model T650SC (Wilsonville, Oreg.) was used to measure the temperature of both ipsilateral and contralateral paws throughout the duration of the experiment immediately after grimace and before von Frey measurements. This was to ensure filament or laser application did not have a direct effect on paw temperature. The temperature across the inflamed plantar paw region was recorded at each time point and analyzed by a blind observer. (Moy et al., 2018a).

Western blots. A month after the inflammation and pain parameters were assessed, animals were deeply anesthetized with isoflurane and quickly decapitated. Lumbar spinal cord and dorsal root ganglia (L3-L5) ipsilateral to the site of injection were extracted from the animals and snap-frozen in liquid nitrogen before being stored at −80° C. These tissues were homogenized in protein extraction buffer (50 mM Tris, 150 mM NaCl, 1 mM EDTA, 0.5% Triton X-100, and 1% SDS). Protease inhibitor cocktail (Sigma P8340), phosphatase inhibitor cocktail 1 (Sigma P2850), and phosphatase inhibitor cocktail 2 (Sigma P5726) were added at 1% final concentration to the protein extraction buffer immediately before addition of buffer to tissue samples. Tissue was homogenized by sonication at 30 Hz for 20-40 s until no clumps remained followed by centrifugation at 13000×g for 15 min at 4° C. The clarified supernatant was used for western blotting. Total protein was estimated using the BCA protein Assay Reagent Kit (Pierce 23,223). Fifteen micrograms of protein was loaded per lane on 12% resolving, 4% stacking polyacrylamide gels. Proteins were transferred onto 0.22 μM nitrocellulose membranes and blocked with 5% nonfat dry milk. Membranes were probed with primary antibodies to TNFα (1:500, Abcam ab1793), IL-1β (1:1000, Abcam ab9722), phosphorylated eIF4E (1:1000, Abcam ab76256), total eIF4E (1:1000, Cell Signaling Technologies 9742), and β-actin (Cell Signaling Technologies 8H10D10, 1:3000) at 4° C. overnight followed by 3 washes with tris-buffered saline (50 mM Tris, 150 mM NaCl, 0.05% Tween-20—TBST), and 1 h incubation with horse radish peroxidase-conjugated secondary antibodies. Membranes were washed three more times using TBST, and target proteins detected via chemiluminescence. Levels were normalized according to β-actin signal.

Statistical analyses. GraphPad Prism software version 8.4 was used for all statistical analysis. For behavior tests, to determine differences between genotypes (WT and eIF4E^{S209A}), two-way repeated measures ANOVA was used for young and aged cohorts, followed by Sidak's post hoc analysis.

For comparing all four cohorts, effect size was calculated by taking the difference of measures after CFA injection at every time point and baseline. Two-way ANOVA was used to analyze the generated values, followed by Tukey's post hoc test for multiple comparisons. Western blot analyses were performed in Image Lab software from BioRad. Target band intensities were normalized according to intensity value changes for β-actin. Two-way ANOVA was used for comparing groups based on age and genotypes followed by Tukey's post hoc. All data are graphically represented by the mean and standard error of the mean. A p value <0.05 was considered significant.

### Results

Validation of eIF4E^{S209A} genotypes and eIF4E protein in all groups. After breeding animals in-house, the inventor checked the genotypes for successful mutants using clipped tails. A representative genotyping gel for WT, eIF4E^{S209A+/-} or heterozygous, and eIF4E^{S209A-/-} or homozygous mutant genotypes is shown in FIG. 2b. The inventor only used homozygous mutants compared with WT animals for all of the experiments. The inventor checked phosphorylated eIF4E in ipsilateral DRGs and spinal cord for all four groups (FIG. 2c representative image). The young and aged eIF4E^{S209A} animals showed no presence of phosphorylated eIF4E, which was clearly seen in both young and aged WT groups. The inventor also confirmed that the expression of total eIF4E protein in the spinal cord and ipsilateral DRGs did not change with age or genotype (FIG. 2d).

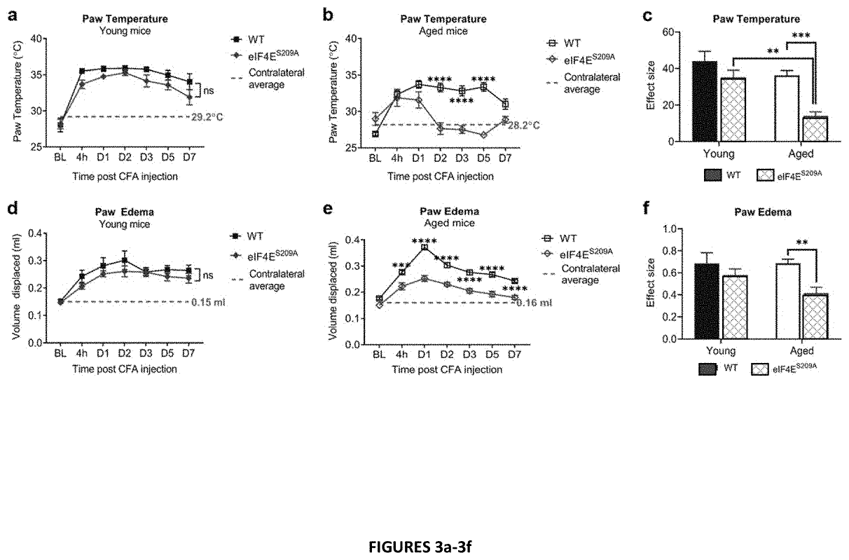

Aged eIF4E^{S209A} mice show faster resolution of inflammation at the injection site. The inventor assessed inflammation at the injection site over 7 days after CFA injection by recording paw temperatures with an infrared camera and measuring paw edema by a plethysmometer. Young mice, both WT and eIF4E^{S209A}, showed consistently elevated paw temperatures and edema across 7 days, indicating ongoing inflammation in their ipsilateral paws (FIGS. 3a and 3b). Aged mice showed a decrease in ipsilateral paw temperature and edema, comparable with contralateral paw readings on day 7 after CFA injection; however, both were still higher than baseline (FIGS. 3a and 3b). Conversely, the aged eIF4E^{S209A} mice had decreased ipsilateral paw temperature and edema starting day 2 post-injection, indicating that inflammation had reduced (FIGS. 3a and 3b). When the magnitudes of differences were compared for all four groups, the aged eIF4E^{S209A} mice had significantly lower temperatures and reduced edema compared with young eIF4E^{S209A} mice and aged WT (FIGS. 3a and 3b) and significantly less edema than aged WT mice. These results demonstrate that eIF4E phosphorylation differentially contributes to inflammation resolution in young versus old mice.

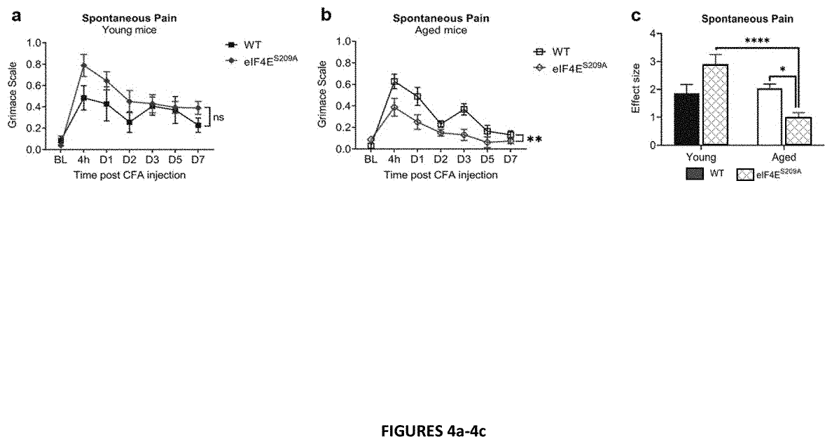

Aged eIF4E^{S209A} mice have a higher threshold for spontaneous pain. The inventor then checked for spontaneous pain over 7 days post CFA injection in the four groups using the grimace scale to score grimacing behavior. The young WT and eIF4E^{S209A} groups did not differ significantly from one another in that they both showed grimacing (FIG. 4a). The aged WT mice showed less grimacing on days 5 and 7, indicating reduced spontaneous pain (FIG. 4b). The aged eIF4E^{S209A} group showed a trending decrease in levels of grimacing across the experiment (FIG. 4b). When the magnitudes of differences for all four groups were compared, an age-dependent effect of eIF4E phosphorylation was seen (Fig. 4c). From this data, the inventor suggests that eIF4E phosphorylation may contribute to spontaneous pain more strongly in aged mice.

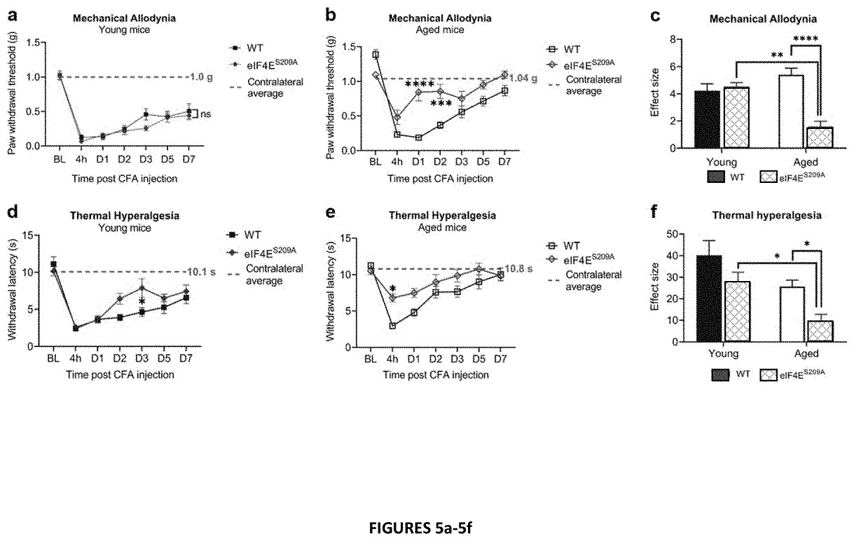

Aged eIF4E^{S209A} mice show reduced mechanical allodynia and less thermal hyperalgesia in response to CFA. Next, the inventor examined the response to evoked pain in all four groups using von Frey filaments for mechanical allodynia and the Hargreaves device for thermal hyperalgesia. Both young WT and eIF4E^{S209A} mutant mice showed mechanical allodynia and thermal hyperalgesia in response to CFA injection (FIGS. 5a and 5d). The aged WT mice showed the same pattern as their young counterparts (FIG. 5 a-e); however, the aged eIF4E^{S209A} mice showed reduced mechanical allodynia and thermal hyperalgesia compared with WT animals (FIGS. 5b and 5e). Interestingly, when the magnitudes of differences for all four groups were compared, the aged eIF4E^{S209A} group was significantly different from the young mutant group and the aged WT group for mechanical allodynia and thermal hyperalgesia (FIGS. 5c and 5f). The inventor therefore concludes that, as shown for spontaneous pain, eIF4E phosphorylation contribution to evoked pain behavior is stronger in aged mice.

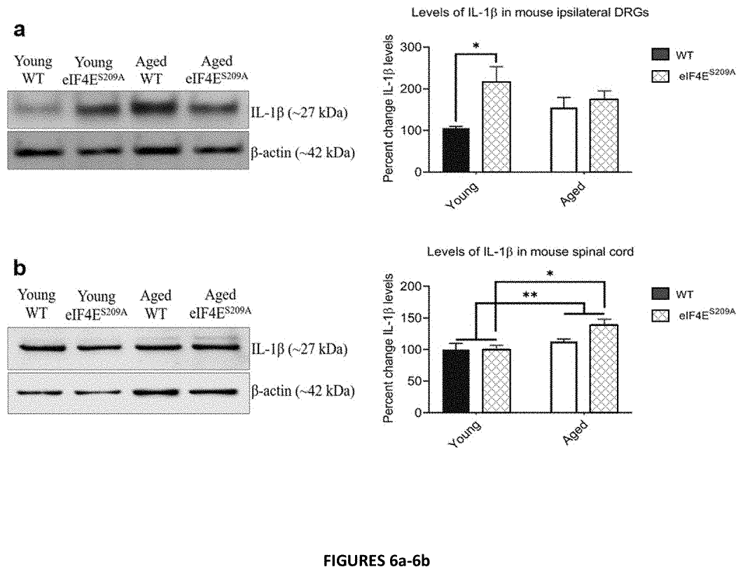

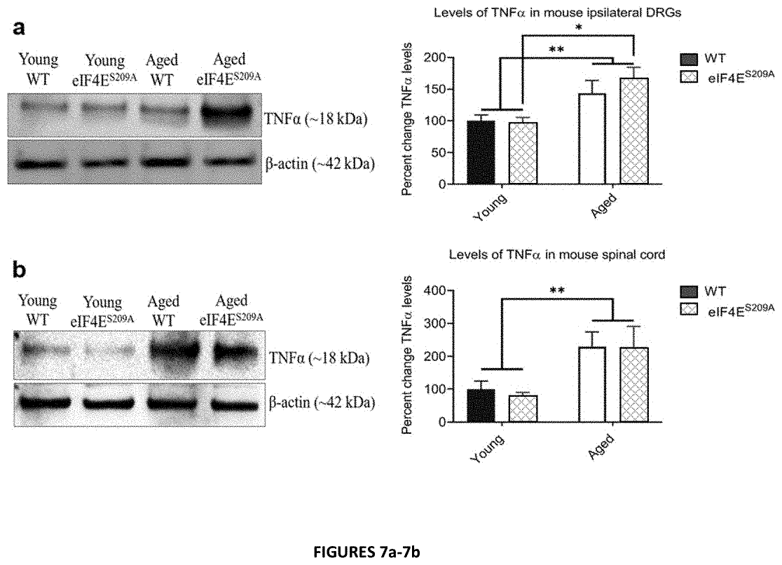

Levels of inflammatory mediators vary based on age and genotype. The inventor examined the levels of pro-inflammatory cytokines IL-1β (FIGS. 6a-b) and TNFα (FIGS. 7a-b) in the ipsilateral DRGs and the spinal cord, 30 days post CFA injection. The inventor found that the aged mice (both WT and eIF4E^{S209A}) had elevated IL-1β (FIG. 6a) and TNFα (FIG. 7a) in their ipsilateral DRGs and spinal cord, respectively. The inventor found no difference between cytokine levels in aged WT mice and those lacking eIF4E phosphorylation. Overall, the aged groups had higher levels of IL-1β and TNFα than their young counterparts which is consistent with an age-associated pro-inflammatory phenotype.

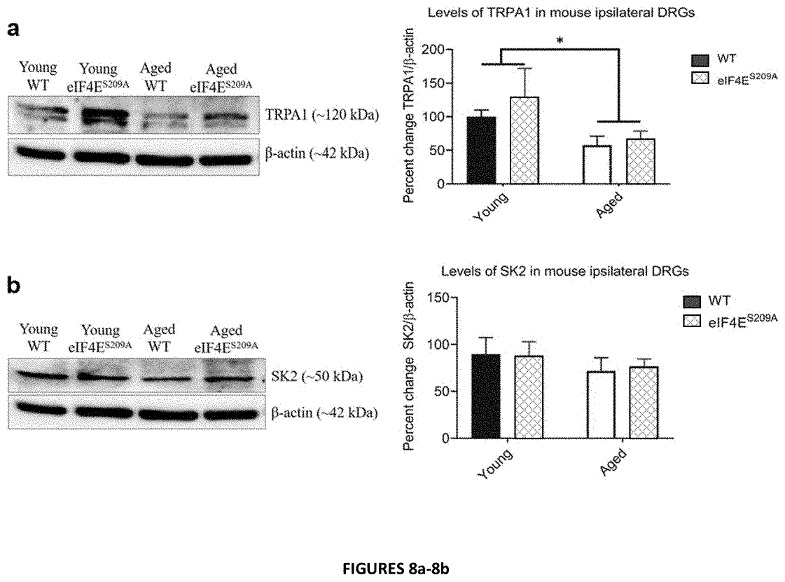

Levels of DRG ion channel proteins change with age. Given the dissociation of higher levels of pro-inflammatory mediators in aging and pain behaviors, the inventor examined the effects of eIF4E phosphorylation on nociceptor plasticity in aging. The inventor probed ipsilateral DRG samples from all four groups for transient receptor potential ankyrin 1 (TRPA1) and small conductance calcium-activated potassium channel 2 (SK2) two cap-dependent ion channels. While the inventor did not find a genotype effect for either protein (FIGS. 8a and 8b), the inventor found that levels of TRPA1 were decreased in samples taken from aged mice (FIG. 8a), indicating that aging may affect ion channel expression for certain TRP channels in the DRG.

### Discussion

The inventor found that in the absence of phosphorylated eIF4E, CFA-induced acute inflammation, spontaneous pain, and mechanical and thermal hypersensitivity are reduced in aged eIF4E mutant mice. However, the levels of pro-inflammatory cytokines IL-1β and TNFα in the ipsilateral DRGs and spinal cord were comparable with aged WT animals. Similar to previous studies, the young mutant mice did not differ significantly in assessed pain behaviors, but the inventor reports inflammatory protein levels in specific peripheral nervous system tissue and observed a genotype effect in the levels of IL-1β in their ipsilateral DRGs compared with young WT mice. However, the aged mutant mice showed a clear increase of IL-1β in the spinal cord and TNFα in ipsilateral DRGs. This data suggests that loss of eIF4E phosphorylation modulates the levels of different pro-inflammatory cytokines, which is dependent upon tissue type. Interestingly, how these inflammatory molecules engender changes in pain behavior in the aged may be less relevant as nociceptor plasticity takes over.

The aged mutants demonstrated faster recovery from CFA-induced inflammatory insult across 7 days when compared with their WT counterparts and young mice of either genotype (FIGS. 3a-f). It has been documented that young adult mice lacking eIF4E phosphorylation show decreased pain responses in hyperalgesic priming, a model of the transition to chronic pain (Moy et al., 2017; Moy et al., 2018b). These mice also show blunted spontaneous pain immediately after inflammatory injury, as measured by paw guarding, but no change in acute peak inflammatory responses (Moy et al., 2018a), consistent with these results. The inventor's data adds novel perspectives to the eIF4E phosphorylation literature revealing that inflammatory responses are attenuated and resolve faster along with spontaneous and evoked pain responses in the aged. To reveal inflammatory mechanisms, the inventor assayed relevant tissue-specific levels of inflammatory mediators and showed they are increased (FIGS. 6a, 6b, 7a and 7b). This suggests that longer-lasting pro-inflammatory cytokine-mediated activation of cells in the peripheral as well as the central nervous system may not be as important for the behavioral effects seen in aging. This leads us to ascertain that eIF4E phosphorylation-dependent translation may play a crucial role in nociceptor plasticity during aging and that this nociceptor plasticity supersedes inflammatory mediators in terms of pain outcomes. To delve into this possibility, the inventor probed DRG tissue for TRPA1 and SK2, ion channels implicated in neuron plasticity, and aging. Interestingly, there is an age-dependent decrease in TRPA1 levels that could explain some of the inventor's observations, but further investigation is needed (FIGS. 8a-b).

This work builds on previous research showing that eIF4E phosphorylation is involved in pain behavior (Moy et al., 2017; Moy et al., 2018b). The present findings illustrate that this pathway plays an even more important role in inflammation and inflammatory pain in aged subjects.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Aguilar-Valles, et al., Translational control of depression-like behavior via phosphorylation of eukaryotic translation initiation factor 4E. Nat Commun. 2018; 9(1):1-14.
Amorim I S, Lach G, Gkogkas C G. The role of the eukaryotic translation initiation factor 4E (eIF4E) in neuropsychiatric disorders. Front Genet. 2018a; 9:561. Published 2018 Nov. 23. //doi.org/10.3389/fgene.2018.00561.
Amorim I S, Kedia S, Kouloulia S, Simbriger K, Gantois I, Jafarnejad S M, et al., Loss of eIF4E phosphorylation engenders depression-like behaviors via selective mRNA translation. J Neurosci. 2018b; 38(8):2118-33.
Burton M D, Johnson R W. Interleukin-6 trans-signaling in the senescent mouse brain is involved in infection-related deficits in contextual fear conditioning. Brain Behav Immun. 2012; 26(5):732-8.
Buxade M, Parra-Palau J L, Proud C G. The Mnks: MAP kinase-interacting kinases (MAP kinase signal-integrating kinases). Front Biosci. 2008; 13:5359-73.
Chaplan S R, Bach F W, Pogrel J W, Chung J M, Yaksh T L. Quantitative assessment of tactile allodynia in the rat paw. J Neurosci Methods. 1994; 53(1).
Chung H Y, Kim D H, Lee E K, Chung K W, Chung S, Lee B, et al., Redefining chronic inflammation in aging and age-related diseases: proposal of the senoinflammation concept. Aging Dis. 2019; 10(2):367-82.
Cruz-Almeida Y, Aguirre M, Sorenson H L, Tighe P, Wallet S M, Riley J L III. Age differences in cytokine expression under conditions of health using experimental pain models. Exp Gerontol. 2015; 72:150-6.
Dahlhamer J, Lucas J, Zelaya C, et al., Prevalence of chronic pain and high-impact chronic pain among adults—United States, 2016. MMWR Morb Mortal Wkly Rep. 2018; 67(36)36): 1001-1006. Published 2018 Sep. 14. //doi.org/10.15585/mmwr.mm6736a2.
Dilger R N, Johnson R W. Aging, microglial cell priming, and the discordant central inflammatory response to signals from the peripheral immune system. J Leukoc Biol. 2008; 84(4):932-9.
Ellis A, Bennett D L. Neuroinflammation and the generation of neuropathic pain. Br J Anaesth. 2013; 111(1)1):26-37. //doi.org/10.1093/bja/aet128.
Frank M G, Barrientos R M, Watkins L R, Maier S F. Aging sensitizes rapidly isolated hippocampal microglia to LPS ex vivo. J Neuroimmunol. 2010; 226(1-2):181-4.
Furic L, Rong L, Larsson O, Koumakpayi I H, Yoshida K, Brueschke A, et al., eIF4E phosphorylation promotes tumor-igenesis and is associated with prostate cancer progression. Proc Natl Acad Sci USA. 2010; 107(32):14134-9.
Garner K M, Amin R, Johnson R W, Scarlett E J, Burton M D. Microglia priming by interleukin-6 signaling is enhanced in aged mice. J Neuroimmunol. 2018; 324:90-9.
Gonskikh Y, Polacek N. Alterations of the translation apparatus during aging and stress response. Mech Ageing Dev. 2017; 168:30-6.
Gubbels Bupp M R, Potluri T, Fink A L, Klein S L. The confluence of sex hormones and aging on immunity. Front Immunol. 2018; 99:1269. Published 2018 Jun 4. //doi.org/10.3389/fimmu.2018.01269.
Hargreaves K, Dubner R, Brown F, Flores C, Joris J. A new and sensitive method for measuring thermal nociception in cutaneous hyperalgesia. Pain. 1988; 32(1):77-88. //doi.org/10.1016/0304-3959(88)90026-7.
Herdy B, Jaramillo M, Svitkin Y V, Rosenfeld A B, Kobayashi M, Walsh D, et al., Translational control of the activation of transcription factor NF-κB and production of type I interferon by phosphorylation of the translation factor eIF4E. Nat Immunol. 2012; 13(6):543-50.
Johnson S C, Rabinovitch P S, Kaeberlein M. mTOR is a key modulator of ageing and age-related disease. Nature. 2013; 493(7432):338-45.
Joshi S, Platanias L C. Mnk kinase pathway: cellular functions and biological outcomes. World J Biol Chem. 2014; 5(3):321-33.
Khoutorsky A, Price T J. Translational control mechanisms in persistent pain. Trends Neurosci. 2018; 41(2):100-14.
Kubben N, Misteli T. Shared molecular and cellular mechanisms of premature ageing and ageing-associated diseases. Nature reviews. Mol Cell Biol. 2017; 18(10):595-609. //doi.org/10.1038/nrm.2017.68.
Langford D J, Bailey A L, Chanda M L, Clarke S E, Drummond T E, Echols S, Glick S, Ingrao J, Klassen-Ross T, Lacroix-Fralish M L, Matsumiya L, Sorge R E, Sotocinal S G, Tabaka J M, Wong D, van den Maagdenberg A M, Ferrari M D, Craig K D, Mogil J S. Coding of facial expressions of pain in the laboratory mouse. Nat Methods. 2010; 7(6):447-449. //doi.org/10.1038/nmeth.1455.
Mangold C A, Wronowski B, du M, Masser D R, Hadad N, Bixler G V, Brucklacher R M, Ford M M, Sonntag W E, Freeman W M. Sexually divergent induction of microglial-associated neuroinflammation with hippocampal aging. J Neuroinflammation. 2017; 14(1):141. //doi.org/10.1186/s12974-017-0920-8.
Megat S, Ray P R, Moy J K, Lou T F, Barragán-Iglesias P, Li Y, et al., Nociceptor translational profiling reveals the Ragulator—Rag GTPase complex as a critical generator of neuropathic pain. J Neurosci. 2019; 39(3):393-411.
Merrick W C, Pavitt G D. Protein synthesis initiation in eukaryotic cells. Cold Spring Harb Perspect Biol. 2018; 10(12)12): a033092. //doi.org/10.1101/cshperspect.a033092.
Moy J K, Khoutorsky A, Asiedu M N, Black B J, Kuhn J L, Barragán-Iglesias P, et al., The MNK-eIF4E signaling axis contributes to injury-induced nociceptive plasticity and the development of chronic pain. J Neurosci. 2017; 37(31):7481-99.
Moy J K, Kuhn J L, Szabo-Pardi T A, Pradhan G, Price T J. eIF4E phosphorylation regulates ongoing pain, independently of inflammation, and hyperalgesic priming in the mouse CFA model. Neurobiol Pain. 2018a; 4:45-50.
Moy J K, Khoutorsky A, Asiedu M N, Dussor G, Price T J. eIF4E phosphorylation influences Bdnf mRNA translation in mouse dorsal root ganglion neurons. Front Cell Neurosci. 2018b;12:29.
Norden D M, Godbout J P. Microglia of the aged brain: primed to be activated and resistant to regulation. Neuropathol Appl Neurobiol. 2013; 39(1):19-34.
Perkins A E, Piazza M K, Deak T. Stereological analysis of microglia in aged male and female Fischer 344 rats in socially-relevant brain regions. Neuroscience. 2018; 377:40-52.
Pinho-Ribeiro F, Verri W, Chiu I. Nociceptor sensory neuron-immune interactions in pain and inflammation. Trends Immunol. 2017; 38(1):5-19. //doi.org/10.1016/j.it.2016.10.001.
Price T J, Gold M S. From mechanism to cure: renewing the goal to eliminate the disease of pain. Pain Med. 2018; 19(8):1525-49.
Rea I M, Gibson D S, McGilligan V, McNerlan S E, Alexander H D, Ross O A. Age and age-related diseases: role of inflammation triggers and cytokines. Front Immunol. 2018; 9:586. //doi.org/10.3389/fimmu.2018.00586.
Roberts, A. W., et al., The population 65 years and older in the United States. 2018. Rostock C, Schrenk-Siemens K, Pohle J, Siemens J. Human vs. mouse nociceptors—similarities and differences. Neuroscience. 2018; 387:13-27.
Rowlett R, Chrestensen C A, Nyce M, Harp M G, Pelo J W, Cominelli F, Ernst P B, Pizarro T T, Sturgill T W, Worthington M T. MNK kinases regulate multiple TLR pathways and innate proinflammatory cytokines in macrophages. American journal of physiology. Gastrointest Liver Physiol. 2008; 294(2):G452-9. //doi.org/10.1152/ajpgi.00077.2007.
Scheper G C, van Kollenburg B, Hu J, Luo Y, Goss D J, Proud C G. Phosphorylation of eukaryotic initiation factor 4E markedly reduces its affinity for capped mRNA. J Biol Chem. 2002; 277(5):3303-9.
Shiers S, Mwirigi J, Pradhan G, Kume M, Black B, Barragan-Iglesias P, Moy J K, Dussor G, Pancrazio J J, Kroener S, Price T J. Reversal of peripheral nerve injury-induced neuropathic pain and cognitive dysfunction via genetic and tomivosertib targeting of MNK. Neuropsychopharmacology. 2020; 45(3):524-533. //doi.org/10.1038/s41386-019-0537-y.

Thoreen C C, Chantranupong L, Keys H R, Wang T, Gray N S, Sabatini D M. A unifying model for mTORC1-mediated regulation of mRNA translation. Nature. 2012; 485(7396):109-13.
Ueda T, Watanabe-Fukunaga R, Fukuyama H, Nagata S, Fukunaga R. Mnk2 and Mnk1 are essential for constitutive and inducible phosphorylation of eukaryotic initiation factor 4E but not for cell growth or development. Mol Cell Biol. 2004; 24(15):6539-49. //doi.org/10.1128/MCB.24.15.6539-6549.2004.
Ye S M, Johnson R W. Increased interleukin-6 expression by microglia from brain of aged mice. J Neuroimmunol. 1999; 93(1-2):139-48.

What is claimed:

1. A method of reducing inflammatory pain in a subject having age-associated inflammation, comprising administering to said subject an amount of a therapeutic sufficient to reduce said inflammatory pain, wherein the therapeutic effectively blocks eIF4E phosphorylation.

2. The method of claim 1, wherein the therapeutic targets MNK to block eIF4E phosphorylation.

3. The method of claim 1, wherein the inflammatory pain includes low-grade inflammation.

4. The method of claim 1, wherein the inflammatory pain includes CFA-induced acute inflammation, spontaneous pain, mechanical hypersensitivity, thermal hypersensitivity, or a combination thereof.
